SEQUENCE LISTING (10 sequences)

SEQ ID NO: 1 - DNA, 20 bp, ARTIFICIAL SEQUENCE
  PRIMER
ttctgtccctttcactcact

SEQ ID NO: 2 - DNA, 18 bp, ARTIFICIAL SEQUENCE
  PRIMER
cctcttctgccagttcca

SEQ ID NO: 3 - DNA, 24 bp, ARTIFICIAL SEQUENCE
  PRIMER
aaagaagggttcccaattaaagat

SEQ ID NO: 4 - DNA, 22 bp, ARTIFICIAL SEQUENCE
  PRIMER
gcattgagagatggactgttag

SEQ ID NO: 5 - DNA, 18 bp, ARTIFICIAL SEQUENCE
  PRIMER
gactgatgctggtgacaa

SEQ ID NO: 6 - DNA, 21 bp, ARTIFICIAL SEQUENCE
  PRIMER
gtgaagtggtatagacaggtc

SEQ ID NO: 7 - DNA, 17 bp, ARTIFICIAL SEQUENCE
  PRIMER
aacgcttcattccaatg

SEQ ID NO: 8 - DNA, 18 bp, ARTIFICIAL SEQUENCE
  PRIMER
ggctctgttgaggtctaa

SEQ ID NO: 9 - DNA, 18 bp, ARTIFICIAL SEQUENCE
  PRIMER
tccaaggagtaagaaacc

SEQ ID NO: 10 - DNA, 18 bp, ARTIFICIAL SEQUENCE
  PRIMER
gaaattgtgagggagatg

(19) United States
(12) Patent Application Publication
(10) Pub. No.: US 2017/0152285 A1
(43) Pub. Date: Jun. 1, 2017

(54) EXTRACTING METHOD OF KUGUACIN, PHARMACEUTICAL COMPOSITION COMPRISING THE KUGUACIN AND USE THEREOF

(71) Applicants: National Taiwan Normal University, Taipei City (TW); Chang Gung Memorial Hospital, Keelung, Keelung City (TW)

(72) Inventors: Po-Jung Tsai, Taipei (TW); Wen-Cheng Huang, Taipei (TW); Tzung-Hsun Tsai, Taipei (TW); How-Ting Ying, Taipei (TW); Ya-Ling Hung, Taipei (TW)

(73) Assignees: National Taiwan Normal University, Taipei City (TW); Chang Gung Memorial Hospital, Keelung, Keelung City (TW)

(21) Appl. No.: 15/284,679

(22) Filed: Oct. 4, 2016

(30) Foreign Application Priority Data
Nov. 27, 2015 (TW) 104139626

(51) Int. Cl. C07J 71/00; A61K 36/42; A61K 31/575; C07J 9/00; A61K 31/58

(52) U.S. Cl. CPC C07J 71/0005; C07J 9/00; A61K 31/58; A61K 31/575; A61K 36/42; A61K 2236/39

(57) ABSTRACT

The present invention is related to an extracting method of kuguacin, pharmaceutical composition comprising the kuguacin and use thereof, specifically related to a use of the kuguacin for treating periodontal diseases.

### CROSS-REFERENCE TO RELATED APPLICATION

This application claims foreign priority under 35 U.S.C. §119(a) to Patent Application No. 104139626, filed on Nov. 27, 2015, in the Intellectual Property Office of Ministry of Economic Affairs, Republic of China (Taiwan, R.O.C.), the entire content of which Patent Application is incorporated herein by reference.

### BACKGROUND OF THE INVENTION

### 1. Field of the Invention

The present invention is related to an extracting method of kuguacin, pharmaceutical composition comprising the kuguacin and use thereof, specifically related to a use of the kuguacin for treating periodontal diseases.

### 2. Description of Related Art

Periodontal diseases are chronic oral inflammatory diseases. The course of periodontal diseases is identified as gingivitis and periodontitis by whether a periodontal pocket exists or not. The major characteristics of gingivitis are bleeding gums and swollen gums. If the gums are constantly under chronic inflammatory condition without improvement, this would eventually cause irreversible periodontitis. When mild periodontitis causes destruction of alveolar bone in the gums and the periodontal tissue, the gum and the tooth therein would separate from each other gradually and form a gap. This pathogenesis condition is referred as a periodontal pocket. Once the periodontal pocket is formed, it easily allows bacteria to grow and food debris to accumulate which may further induce the destruction of periodontal ligament, the course thereof is referred as moderate periodontitis. Severe periodontitis causes large loss of bone substance around alveolar bone which may result in symptoms such as gingival recession (receding gums), exposed tooth root, tooth loss, and so on [1, 2].

One of the main causes of periodontal diseases is the large growth of pathogenic bacteria in oral cavity. When the pathogenic bacteria invade periodontal tissue, polymorphonuclear leukocyte and macrophage would be triggered to secrete cytokine, chemokine and adhesion molecule. It would transfer immunocytes toward the infectious sites to produce various of inflammatory mediators which could lead to vascular dilatation, cell infiltrating, interstitial fluid leakage and the like. Therefore, in the long run, periodontal diseases cause difficulty of mastication in daily diet and the intrusion of large number of bacteria in bloodstream leading to bacteremia. It further affects the function of body tissues and organs, which places the subject (such as mammals including rodents and primates, e.g., mice and human) at more serious risk of systemic diseases, for example:

1. Cardiovascular diseases: Inflammation is one of the factors that can contribute to hardening of the arteries. The chronic inflammation caused by periodontitis plays a vital role in the development of coronary artery disease. When a subject suffers from coronary artery disease, it can be found that the concentrations of C-reactive protein (CRP), fibrinogen, and cytokines would increase in the subject. Regarding periodontitis, the concentrations of aforesaid proteins and cytokines in the peripheral circulation are increasing as well [3]. Besides, the pathogenic bacteria of periodontitis would enhance the activity of platelets to induce blood coagulation, increase the formation of plaques and develop atherosderosis [4].

2. Respiratory tract diseases: Poor oral hygiene may cause periodontitis which is also a risk factor for inducing pulmonary diseases. This may result from the symbiosis of the bacteria causing lung infection and oral bacteria, which would form biofilms that cause dental plaques and dental calculus. They are considered as hotbeds of bacteria [5].

3. Diabetes: Diabetes and periodontitis disease have mutual influences. Diabetes would increase the incidence of periodontitis disease and enhance its progression. The infection of gum may cause poor blood glucose control for a subject suffering from diabetes. On the contrary, if periodontitis has been treated properly, then blood glucose can be controlled effectively, and thus lower the risk of development of other complications led by diabetes [6].

4. Unfavorable pregnancy outcome: Premature delivery and low birth weight of infant often happen to pregnant women who suffered from periodontitis. Recently, it discovers that the weakened immune function of pregnant women who suffered from respiratory tract infection due to periodontitis may lead to the transfer of the pathogenic periodontitis bacteria to their placentas through blood circulation, which may pose a risk of stillbirth [7].

Porphyromonas gingivalis (P. gingivalis) is a gram-negative anaerobic bacterium. The morphology thereof is black and rod-like. It is one of the pathogenic bacteria of chronic periodontitis.

P. gingivalis modulates the development of inflammation mainly via the activation of downstream signal transduction pathways, such as MyD88, mitogen-activated protein kinase (MAPK), nuclear factor-κB (NF-κB) and the like by the toll-like receptor 2 (TLR2) of endothelial cells and immune cells. The activated cells release large amount of pro-inflammatory cytokines such as interleukin-6 (IL-6), IL-8, IL-1β, and tumor necrosis factor-α (TNF-α). This results in chronic inflammation in periodontal tissue, and thus poses higher risk of periodontitis [8-10].

The treatments for periodontal diseases/periodontitis can be classified as medication therapies and surgical treatments. Medication therapies aim at anti-inflammation, whereas surgical treatments are to remove the pathogenic factors like dental plaques and dental calculus in periodontal pockets using mechanical apparatus and to enhance the proliferation of injured tissue by bioengineering [11].

The medication therapies are predominantly based on antibiotics to inhibit the formation of dental plaques in reducing the incidence of gingivitis. Medicaments often used include non-steroid anti-inflammatory drugs (NSAIDs), doxycycline [12, 13] and anti-bacterial mouthwash containing chlorhexidine. However, a long-term use of these medicaments may lead to some side effects. Regarding NSAIDs, the common adverse side effects include gastrointestinal discomfort, bleeding, central nervous system disorder, inhibition of platelet agglutination, allergic reaction and so on.

According to the aforementioned, scaling dental calculus and dental plaques on teeth using mechanical apparatus or administering antibiotics is the primary clinical treatment for periodontal diseases/periodontitis. However, when periodontitis is too severe, the gum cannot be recovered to its healthy state. Therefore, under the considerations of the side effects of the medicaments and the drug resistance of microorganisms, a method for preventing and treating periodontitis or related inflammatory diseases caused by P. gingivalis is needed.

### SUMMARY OF THE INVENTION

In view of the above-described issues, the present invention finds that kuguacin extracted from Momordica charantia L., a cucurbitane triterpenoids like compound, is effective in reducing the inflammation caused by P. gingivalis and can be applied to the prevention and treatment of periodontitis.

The present invention provides an extracting method for extracting kuguacin from Momordica charantia, comprising:

providing powder of M. charantia leaves;

extracting a pre-determined weight of the powder of M. charantia leaves with an extracting agent to obtain crude extract thereof;

subjecting the crude extract to a partition extraction process with a first elution solution by column chromatography to obtain a first fraction, wherein the first elution solution is n-hexane or a mixture of more than 0% to 30% by volume of ethyl acetate and 70% to less than 100% by volume of n-hexane;

subjecting the first fraction to a partition extraction process with a second elution solution by column chromatography to obtain a second fraction; and

subjecting the second fraction to a partition extraction process with a third elution solution by column chromatography to obtain a third fraction.

According to an aspect of the present invention, the extracting agent comprises at least one selected from a group consisting of methanol, ethanol and ethyl acetate.

In an embodiment of the present invention, the second elution solution is n-hexane or a mixture of more than 0% to 50% by volume of acetone and 50% to less than 100% by volume of n-hexane.

In an embodiment of the present invention, the third elution solution is a mixture of 20% to 80% by volume of n-hexane and 20% to 80% by volume of acetone.

In another embodiment of the present invention, the method further comprising subjecting the third fraction to a partition extraction process with a fourth elution solution by column chromatography to obtain a fourth fraction. In one embodiment, the fourth elution solution is n-hexane or a mixture of 50% to less than 100% of n-hexane and more than 0% to 50% of acetone.

According to the method of the present invention, the kuguacin extracted is 5β,19-epoxycucurbita-6,23(E),25(26)-triene-3β,19(R)-diol, 5,19-epoxycucurbita-6,23-diene-3,19,25-triol or 3,7,25 -trihydroxycucurbita-5,23-dien-19-al.

In another aspect, the present invention provides a pharmaceutical composition comprising a therapeutically effective amount of kuguacin obtained from the method of the present invention; and a pharmaceutical acceptable carrier.

In another aspect, the present invention provides a use of kuguacin for the manufacture of a medicament for preventing or treating periodontal diseases. For example, the present invention provides a use of kuguacin extracted by the method of the present invention for the manufacture of a medicament for preventing or treating periodontal diseases.

The present invention further provides a use of kuguacin for the manufacture of a medicament for preventing or treating diseases caused by Porphyromonas gingivalis.

For example, the present invention provides a use of kuguacin extracted by the method of the present invention for the manufacture of a medicament for preventing or treating diseases caused by Porphyromonas gingivalis.

In one embodiment of the present invention, the diseases caused by P. gingivalis are gingivitis, periodontitis and/or cardiovascular diseases.

The method provides by the present invention can manufacture large quantities of kuguacin. Besides, the present invention finds that the kuguacin can effectively inhibit inflammation caused by P. gingivalis.

### BRIEF DESCRIPTIONS OF THE DRAWINGS

FIG. 1 shows a flowchart of an extraction process for obtaining extracts from Momordica charantia;

FIG. 2 shows the effects of different M. charantia extracts on the viability of THP-1 cell line. The THP-1 cell lines were treated by different M. charantia extracts for 24 hours and the control group (ctrl) was untreated. The data is represented by means±standard deviation (n=3);

FIGS. 3A to 3I show the effects of different M. charantia extracts on the expressions of cytokines, IL-8 and IL-6 by THP-1 cell lines induced by P. gingivalis. The THP-1 cell lines were co-cultured with P. gingivalis (MOI=10) and DMSO, different M. charantia extracts or luteolin (10 μM) for 24 hours. The control group (ctrl) was untreated by P. gingivalis and the extracts. The data is represented by means±standard deviation (n=3);

FIGS. 4A to 4E show the effects of different M. charantia extracts on the phosphorylation of signal transduction proteins in THP-1 cell lines induced by P. gingivalis. FIG. 4A shows the levels of p-ERK, p-JNK and p-p38 in THP-1 cell lines induced by P. gingivalis at different time points; FIGS. 4B to 4E show effects of fractions 5211, 5212 and 532 on the levels of p-ERK, p-JNK and p-p38 in THP-1 cell lines induced by P. gingivalis for 30 minutes. The control group (ctrl) was untreated by P. gingivalis and the extracts; p-ERK, p-JNK and p-p38 represent phosphorylated ERK, phosphorylated JNK and phosphorylated p38, respectively; t-ERK, t-JNK and t-p38 represent total ERK, total JNK and total p38, respectively;

FIG. 5 shows a flowchart for the experiment of animal model of periodontitis;

FIGS. 6A to 6F show in the model of periodontitis, the effects of different M. charantia extracts on the m-RNA expression levels of IL-6, COX-2, TNF-α and iNOS in the gum tissue induced by P. gingivalis (P.g). The vehicle group and the control group were untreated by P. gingivalis and the extracts.

### DETAILED DESCRIPTION OF THE INVENTION

The present invention is described by using the following embodiments, so as to enable a person skilled in the art to conceive the other advantages and effects of the present invention from the invention of the present specification. However, the examples in the present invention are not used for limiting the scope of the present application. Any one skilled in the art can alter or modify the present invention in any way, without departing from the spirit and scope thereof. Therefore, the scope of the present invention should be accorded with the definitions in the appended claims.

It should be noted that the singular forms “one” and “the” used in the present specification include plural forms too, unless clearly and definitively limit to one specific form. Unless clearly indicated in the context, the terms “or” and “and/or” are used interchangeably. At the same time, the terms, such as “first”, “second”, “third” and “fourth,” used in the present specification are merely for enhancing the understanding of the descriptions, rather than limit the implementable scope of the present disclosure. Without materially altering the technical content, the alteration or adjustment of relative relationships should also be regarded as fallen within the implementable scope of the present disclosure.

The present invention is related to an extracting method of kuguacin, a pharmaceutucal composition comprising the kugiacin, and use thereof.

It has been reported that bitter gourd has several curative effects, including anti-diabetes, anti-inflammation, anti-tumor, etc. There are many active ingredients in bitter gourd, which can be obtained through extraction from different parts of the bitter gourd plant, such as fruits, leaves, seeds, and stems. The compounds purified from bitter gourd include tetracyclic cucurbitane terpenoids, saponins, steroid compounds and alkaloids, wherein tetracyclic cucurbitane terpenoids are the majority, which are also referred as kuguacin.

The present invention provides an extracting method for obtaining kuguacin from Momordica charantia, comprising:

providing powder of M. charantia leaves;

extracting a pre-determined weight of the powder of M. charantia leaves with an extracting agent to obtain a crude extract thereof;

subjecting the crude extract to a partition extraction process with a first elution solution by column chromatography to obtain a first fraction, and wherein the first elution solution is n-hexane or a mixture of more than 0% to 30% by volume of ethyl acetate and 70% to less than 100% by volume of n-hexane;

subjecting the first fraction to a partition extraction process with a second elution solution by column chromatography to obtain a second fraction; and

subjecting the second fraction to a partition extraction process with a third elution solution by column chromatography to obtain a third fraction.

In the method of the present invention, water, acids, bases, buffers, organic solvents or mixture thereof can be used as the extracting agent. The acids may be various of organic acids or inorganic acids; the bases may be various of organic bases or inorganic bases; the buffers may be various of buffered salt solutions; the organic solvents may be various of organic solvents. Preferably, the extracting agent is water, acids, organic solvents miscible with water or mixture thereof.

According to the embodiments of the present invention, the extracting agent may be alcohols (such as methanol and ethanol), ethyl acetate or mixture thereof. In another embodiment, the extracting agent may further comprise acids. For example, the extracting agent may be the mixture of methanol and hydrogen chloride.

According to the embodiments of the present invention, the ratio of the powder of M. charantia leaves and the extracting agent may be in the range of 1:20 to 5:1 (w/v).

In the method of the present invention, water, buffer, organic solvents, or mixture thereof can be used as elution solution. The organic solvents may be alkanes, alcohols, ketones, aldehydes, esters, etc. Preferably, the elution solution may be a single organic solvent or a mixture of multiple organic solvents. Preferably, the elution solution may be a mixture of two or more solvents having various differences in polarities (polarity-alterable) or isotonicities.

The terms used herein have the same meaning as commonly understood by one of ordinary skill in the art to which this invention belongs. The term “isotonic” refers to a constant mixing ratio of the elution solutions during the operation of the column chromatography method. The term “polarity-alterable” refers to mixing two or more solutions having different polarities as elution solutions, and the mixing ratio of the elution solutions can be altered from one ratio to another ratio during the step of the separation of the fractions in order to change the polarity of the elution solution. The changes of polarity can be a gradient variation, but not limited to it.

According to the embodiments of the present invention, the elution solutions may be n-hexane, ethyl acetate, acetone or mixture thereof. For example, the mixture may be but not limited to a polarity-alterable mixture or an isotonic mixture of n-hexane and ethyl acetate, a polarity-alterable mixture or an isotonic mixture of n-hexane and acetone, acetone, etc.

It should be noted that, the “first” elution solution used herein refers to an elution solution which can be used to separate the “first” fraction in the step of separating the “first” fraction, which is not intended to define it as a composition with a single ratio. Likewise, the “second” elution solution, the “third” elution solution and the “fourth” elution solution refer to those which can be used to separate the “second”, the “third” and the “fourth” fractions, respectively, which are not intended to define them as compositions with a single ratio.

For example, according to the embodiments of the present invention, when the first elution solution is a polarity-alterable mixture of n-hexane and ethyl acetate, in the step of separating the first fraction, the ratio of the first elution solution may be altered in a stepwise way, such as gradually adjusting the ratio from 100% of n-hexane to 5% of ethyl acetate/95% of n-hexane, to 10% of ethyl acetate/90% of n-hexane, to 15% of ethyl acetate/85% of n-hexane, to 20% of ethyl acetate/80% of n-hexane, to 25% of ethyl acetate/75% of n-hexane, to 30% of ethyl acetate/70% of n-hexane. All mixtures with the above ratios are referred as the first elution solution. Alternatively, the first elution solution may be an isotonic mixture of n-hexane:ethyl acetate=80:20 (v/v).

When the second elution solution is a polarity-alterable mixture of n-hexane and acetone, in the step of separating the second fraction, the ratio of the second elution solution may be altered in a stepwise way, such as gradually adjusting the ratio from 100% of n-hexane, to 5% of acetone/95% of n-hexane, to 10% of acetone/90% of n-hexane, to 20% of acetone/80% of n-hexane, to 30% of acetone/70% of n-hexane, to 40% of acetone/60% of n-hexane, to 50% of acetone/50% of n-hexane, to 60% of acetone/40% of n-hexane, to 70% of acetone/30% of n-hexane, to 80% of acetone/20% of n-hexane. All mixtures with the above ratios are referred as the second elution solution. Alternatively, the second elution solution may be an isotonic mixture of n-hexane:acetone=50:50 (v/v).

When the third elution solution is a polarity-alterable mixture of n-hexane and acetone, in the step of separating the third fraction, the ratio of the third elution solution may be altered in a stepwise way, such as gradually adjusting the ratio from 20% of acetone/80% of n-hexane, to 30% of acetone/70% of n-hexane, to 40% of acetone/60% of n-hexane, to 50% of acetone/50% of n-hexane, to 60% of acetone/40% of n-hexane, to 70% of acetone/30% of n-hexane, to 80% of acetone/20% of n-hexane. All mixtures with the above ratios are referred as the third elution solution. Alternatively, the third elution solution may be an isotonic mixture of n-hexane:acetone=80:20 (v/v).

In some embodiments, the first elution solution of the method of the present invention is a polarity-alterable mixture or an isotonic mixture of n-hexane and ethyl acetate; the second elution solution is a polarity-alterable mixture or an isotonic mixture of n-hexane and acetone; and the third elution solution is a polarity-alterable mixture or an isotonic mixture of n-hexane and acetone.

According to another embodiment, the first elution solution may be a mixture of n-hexane:ethyl acetate=80:20 (v/v); the second elution solution may be a mixture of n-hexane:acetone=50:50 (v/v); and the third elution solution may be a mixture of n-hexane:acetone=40:60 (v/v).

According to another embodiment, the first elution solution may be a mixture of n-hexane:ethyl acetate=80:20 (v/v); the second elution solution may be a mixture of n-hexane:acetone=50:50 (v/v); and the third elution solution may be a mixture of n-hexane:acetone=80:20 (v/v).

In another embodiment, the third elution solution used in the method of the present invention may be a polarity-alterable mixture or an isotonic mixture of 20% to 80% of acetone and 20% to 80% of n-hexane by volume.

According to the embodiments of the present invention, the method may further comprise subjecting the third fraction to a partition extraction process with a fourth elution solution by column chromatography to obtain a fourth fraction. The fourth elution solution may be a polarity-alterable mixture or an isotonic mixture of 50% to 100% of n-hexane and 0% to 50% of acetone or a mixture of 70% to less than 100% of n-hexane and more than 0% to 30% of acetone.

According to the embodiments of the present invention, when the fourth elution solution is a polarity-alterable mixture of n-hexane:acetone, in the step of separating the fourth fraction, the ratio of the fourth elution solution may be altered in a stepwise way, such as gradually adjusting the ratio from 10% of acetone/90% of n-hexane, to 20% of acetone/80% of n-hexane, to 30% of acetone/70% of n-hexane, to 40% of acetone/60% of n-hexane, to 50% of acetone/50% of n-hexane. All mixtures with the above ratios are referred as the fourth elution solution. Alternately, the fourth elution solution may be an isotonic mixture of n-hexane:acetone=70:30 (v/v).

According to some preferred embodiments, the fourth elution solution may be a mixture of n-hexane:acetone=70:30 (v/v).

According to the embodiments of the present invention, the third fraction obtained by the method provided by the present invention includes kuguacin: 5β,19-epoxycucurbita-6,23(E),25(26)-triene-3β,19(R)-diol (also referred as fraction 532 herein).

According to the embodiments of the present invention, the fourth fraction obtained by the method provided by the present invention includes kuguacin: 5,19-epoxycucurbita-6,23-diene-3,19,25-triol (often referred as fraction 5211 herein) or 3,7,25-trihydroxycucurbita-5,23-dien-19-al (often referred as fraction 5212 herein).

According to the embodiments of the present invention, the kuguacins obtained by the method provided by the present invention have chemical structures shown below:

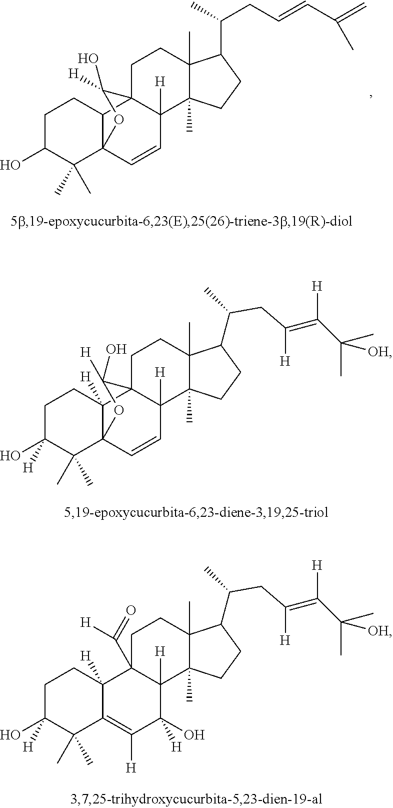

In other aspect, the present invention provides a pharmaceutical composition, comprises a therapeutically effective amount of kuguacin; and a pharmaceutical acceptable carrier.

The kuguacin comprised in the pharmaceutical composition of the present invention can be those extracted by the method of the present invention.

In the pharmaceutical composition of the present invention, the therapeutically effective amount of kuguacin is from 1 μM to 20 μM.

The “pharmaceutical acceptable carrier” used herein means suitable vehicles comprising one or more compatible solid or liquid diluting fillers, excipients, encapsulating materials or adjuvants. All of the vehicles are suitable for topical oral use. In terms of the pharmaceutical composition of the present invention, the term “compatible” used herein means that each of the components in the composition may mixes well with each other without interaction that may reduce the stability and/or the efficacy of the composition.

The dosage form of the pharmaceutical composition of the present invention can comprise tooth paste (includes gel and subgingival gels), mouthwash, mouth sprays, guns, lozenges or dental solution and the like. The terms “lozenges”used herein comprises dental solid formulations such as breath mints, troches, pastilles, microcapsules and the like.

One with ordinary skill in the art can choose suitable components as carriers in need. For example, as described in U.S. Pat. No. 3,988,433, when the pharmaceutical composition is a toothpaste (including dental gel and so on), carriers can be selected from abrasive materials, foaming agents, binders, humectants, flavoring agents, sweetening agents and the like. When it is for mouthwash, carriers can be selected from water, flavoring agents, sweetening agents and the like. When it is a mouth spray, “carriers for mouth spray” may be chosen. Alternately, as described in U.S. Pat. No. 4,083,955, when the pharmaceutical composition is a lozenge, “carriers for lozenges carrier” may be chosen. When it is a chewing gum, “carriers for chewing gum” may be chosen; or for example, as described in U.S. Pat. No. 5,198,220, when the pharmaceutical composition is a subgingival gel, “carriers for subgingival gels” may be chosen. All of the above disclosures are hereby incorporated herein by reference.

The pharmaceutical composition of the present invention may be an oral composition. For example, the pharmaceutical compositions can be dentifrices such as toothpastes, tooth gels, and tooth powders. As described in U.S. Pat. No. 8,277,782, components of such toothpaste and tooth gels generally include one or more of dental abrasives (from about 10% to about 50%), surfactants (from about 0.5% to about 10%), thickening agents (from about 0.1% to about 5%), humectants (from about 10% to about 55%), flavoring agents (from about 0.04% to about 2%), sweetening agents (from about 0.1% to about 3%), coloring agents (from about 0.01% to about 0.5%) and water (from about 2% to about 45%). When the pharmaceutical composition is a tooth powder, it does not contain liquid.

Alternatively, the pharmaceutical composition of the present invention can be a gel or a subgingival gel which includes a thickening agent (from about 0.1% to about 20%), a humectant (from about 10% to about 55%), a flavoring agent (from about 0.04% to about 2%), a sweetening agent (from about 0.1% to about 3%), a coloring agent (from about 0.01% to about 0.5%), and water.

The pharmaceutical composition of the present invention can be a mouthwash or a mouth spray which includes water (from about 45% to about 95%), ethanol (from about 0% to about 25%), a humectant (from about 0% to about 50%), a surfactant (from about 0.01% to about 7%), a flavoring agent (from about 0.04% to about 2%), a sweetening agent (from about 0.1% to about 3%), and a coloring agent (from about 0.001% to about 0.5%).

The pharmaceutical composition of the present invention can be a dental solution which includes water (from about 90% to about 99%), a preservative (from about 0.01% to about 0.5%), a thickening agent (from 0% to about 5%), a flavoring agent (from about 0.04% to about 2%), sweetening agent (from about 0.1% to about 3%), and a surfactant (from 0% to about 5%).

The pharmaceutical composition of the present invention can also be a chewing gum which typically includes one or more of a gum base (from about 50% to about 99%), a flavoring agent (from about 0.4% to about 2%) and a sweetening agent (from about 0.01% to about 20%).

The present invention further provides a use of kuguacin for the manufacture of a medicament.

The medicament comprises kuguacin, which is used to decrease the expression levels of cytokines (such as IL-8, IL-6 and TNF-α), so as to reduce inflammation and to treat periodontal diseases or diseases caused by Porphyromonas gingivalis, wherein the diseases include gingivitis, periodontitis and cardiovascular diseases.

Treating or preventing diseases caused by Porphyromonas gingivalis means that to treat or to prevent oral diseases including gingivitis with the pharmaceutical composition of the present invention or with the manufactured medicament and thereby promoting and enhancing whole body health for the subject being treated which can be evidenced by the following health indices or biomarkers as described in U.S. Pat. No. 8,277,782:

1) reduction in the risk of development of heart attack, stroke, diabetes, severe respiratory infections, low birth weight infants, post-partum dysfunction in neurologic/developmental function, and associated increased risk of mortality;

2) reduction in the development of fatty arterial streaks, atherosclerotic plaques, progression of plaque development, thinning of the fibrous cap on atherosclerotic plaques, rupture of atherosclerotic plaques, and the subsequent blood clotting events;

3) reduction in carotid arterial (intimal) wall thickness (e.g., as assessed by ultra-sound techniques);

4) reduction in exposure of blood and systemic circulation to oral pathogens and/or their toxic components, especially reduction in blood levels of oral bacteria, lipopolysaccharide (LPS) and/or the incidence of oral pathogens and/or components thereof found in arterial plaques, arterial structures, and/or distant organs (e.g., heart, liver, pancreas, kidney);

5) reduction in the exposure of the lower respiratory track to the inhalation of bacterial pathogens and the subsequent development of pneumonias and/or exacerbation of chronic obstructive lung disease;

6) reduction in alterations in circulating hematocrit, hemoglobin, white blood cell count and/or platelet counts;

7) reduction in the incidence of disregulation in blood/serum levels of inflammatory mediators/cytokines such as TNF-α, IL-6, CD-14, and IL-1;

8) reduction in the incidence of disregulation of blood/serum levels of acute phase reactants including C-reactive protein, fibrinogen, α1-antitrypsin, and haptoglobin;

9) reduction in the incidence of disregulation of blood/serum markers of metabolic disregulation including homocysteine, glycosylated hemoglobin, 8-iso-PGF-2α, and uric acid;

10) reduction in incidence of disregulation of glucose metabolism (typically assessed by impaired glucose tolerance test, increased fasting blood glucose levels, and abnormal fasting insulin levels); and 11) reduction in disregulation of blood lipid levels (specifically including blood or serum cholesterol, triglycerides, LDL, HDL, VLDL, Apolipoprotein B, and/or Apolipoprotein A-1).

The pharmaceutical composition of the present invention is capable of treating and preventing diseases or conditions mediated by bacteria existing in oral cavity (e.g. P. gingivalis) effectively, such as dental plaques, gingivitis, periodontitis, etc. The pharmaceutical composition of the present invention can further prevent or treat other diseases caused by the spread of the pathogenic bacteria and related harmful substances into blood stream and other sites in the body by controlling these diseases and conditions.

The effects of the present disclosure are further illustrated by the following specific embodiments, which are not intended to limit the scope of the invention.

### EXAMPLES

### Example 1

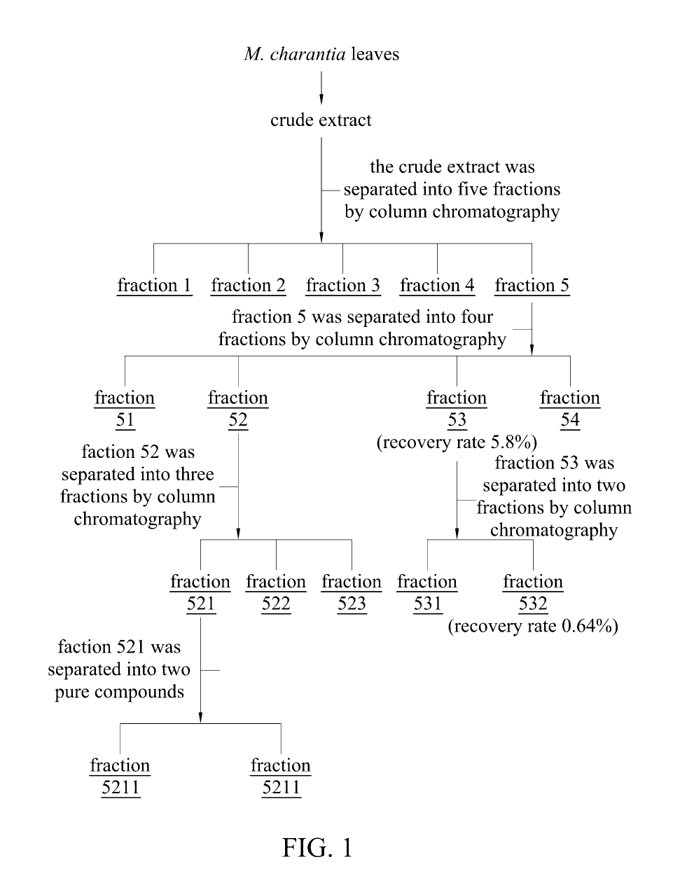

### Referring to FIG. 1

Fresh M. charantia leaves (Variety Hualien No. 1, provided by Mr. Jong-Ho Chyuan in Division of Crop Improvement of Hualien District Agricultural Research and Extension Station) were washed by water and air-dried at room temperature. Then, after dehydrating by a freeze-drying machine, the dehydrated leaves were grounded using high-speed grinder, and stored at −80° C. for the next step.

The powder of M. charantia leaves was extracted by ethanol at a ratio of 1:20 (w/v) at room temperature for 24 hours. Then the mixture was filtered by a Büchner funnel and concentrated under reduced pressure. The steps of extracting, filtering and concentrating under reduced pressure were applied repeatedly to the filtered residues until the weight of the extract no longer increased. Thereby a crude extract was obtained (recovery rate: 12.2%).

The crude extract was re-dissolved and applied to a silica-packed column for partition extraction. When the layers were separated, the first elution solution was used as mobile phase for elution. The filtrate of each layer was collected separately and concentrated. 5 fractions were obtained separately from the crude extract, which were referred as factions 1 to 5, respectively, wherein the recovery rate of fraction 5 was 70.4%. In the present embodiment, the first elution solution was a mixture of n-hexane:ethyl acetate=80:20 (v/v).

The fraction 5 was applied to another open-style column packed with silica and the column was eluted by a second elution solution so that the fraction 5 was layered gradually. The filtrates were collected in order and concentrated to obtain fractions 51 to 54, wherein the recovery rate of the fraction 53 was 5.8%. In the present embodiment, the second elution solution was a mixture of n-hexane:acetone=50:50 (v/v).

The fraction 53 was applied to another open-style column packed with silica and the column was eluted by a third elution solution to obtain a third fraction. In the present embodiment, the third elution solution was a polarity-alterable mixture of n-hexane:acetone. The column was firstly eluted by a mixture of n-hexane and acetone at a ratio of 80:20 (v/v) until the fraction 53 was layered and then was eluted by a mixture of n-hexane and acetone at a ratio of 40:60 (v/v). The proportion of acetone was increased gradually to reach 100% of acetone at the end. By collecting the filtrates in order via the alteration of the polarity of the third elution solution, the filtrates were concentrated under reduced pressure to obtain the third fractions which were referred as fraction 531 and fraction 532, respectively. The recovery rate of the fraction 532 was 0.64%.

The fraction 532 was analyzed by ¹H-NMR and ¹³C-NMR for structure identification. It was found that fraction 532 was kuguacin, 5β,19-epoxycucurbita-6,23(E),25(26)-triene-3β,19(R)-diol.

### Example 2

### Referring to FIG. 1

The powder of M. charantia leaves obtained in Example 1 was used. Five gram of said powder of M. charantia leaves was extracted by the solution of methanol/hydrochloride acid (100:1, v/v). The mixture was centrifuged at 5,000 rpm, and the supernatant was collected and concentrated under reduced pressure (45-50° C.). After redissolving the residues by 25 mL of water/ethanol solution (80:20, v/v), 25 mL of ethyl acetate was used to extract the residues for 4 times to obtain an organic layer which was then dehydrated by anhydrous sodium sulfate for 30 to 40 mins. Then, the organic layer was filtered and dried by vaccuum evapoaration (45-50° C.). A crude extract was obtained.

The crude extract was separated by silica column chromatography and was eluted by a first elution solution to obtain fractions 1 to 5. In the present embodiment, the first elution solution was a mixture of n-hexane:ethyl acetate=80:20 (v/v).

Next, the fraction 5 was fractionated into fractions 51 to 54 by silica column chromatography with a second elution solution as a mobile phase, wherein the recovery rate of the fraction 52 was 15.1%. In the present embodiment, the second elution solution was a mixture of n-hexane:acetone=50:50 (v/v).

The fraction 52 was then fractionated into fractions 521 to 523 by silica column chromatography with a third elution solution as a mobile phase, wherein the recovery rate of the fraction 521 was 74.3%. In the present embodiment, the third elution solution was a mixture of n-hexane:acetone=80:20 (v/v).

The fraction 521 can further be purified and separated by a fourth elution solution to obtain fraction 5211 and fraction 5212. The recovery rates thereof were 4.5% and 13.4%, respectively. In the present embodiment, the fourth elution solution was a mixture of n-hexane:acetone=70:30 (v/v).

The structures of fractions 5211 and 5212 were determined by analysis, such as ¹H-NMR, ¹³C-NMR, HSQC, HMBC and COSY, and named as 5,19-epoxycucurbita-6,23-diene-3,19,25-triol and 3,7,25-trihydroxycucurbita-5,23-dien-19-al, respectively.

### Example 3

### The Effects of the M. charantia Extract on the Viability of THP-1 Cells

### Cell Culture

Human acute monocytic leukemia (THP-1, BCRC 60430) was purchased from Bioresource Collection and Research Center of Food Industry Research and Development Institute in Hsinchu. In the culture medium of THP-1, besides comprising RPMI 1640 medium (Gibco BRL, Grand Island, N.Y., US) and 10% of fetal bovine serum (Gibco) 2 μM of L-glutamine, 4.5 g/L of glucose, 10 μM of HEPES, 1.0 mM of sodium pyruvate (Sigma), 0.05 mM of 2-mercaptoethanol (Sigma) and 1% of Antibiotic-Antimycotic (Gibco) (which including 100 units/mL of penicillin G sodium, 100 μg/mL of streptomycin sulfate and 250 ng/mL of amphotericin B) were added. The cells were placed in an incubator at 37° C. in 5% of CO₂. The medium was changed every 2 or 3 days.

### MTT Assay for Testing Cell Viability

THP-1 cell line (2×10⁶ cells/mL) was inoculated in a 96-well plate with 100 μL culture medium in each well. Different concentrations of the samples from Example 1 or 2 (each of which was 100 μL) were added into the wells as treatment or the culture medium was added as control. After incubating in an incubator at 37° C. in 5% of CO₂ for 24 hours, MTT assay was conducted to determine the viability of THP-1 cells by a formula below:

Viability of cell (%)=the absorbance of the treatment/the absorbance of the control×100%.

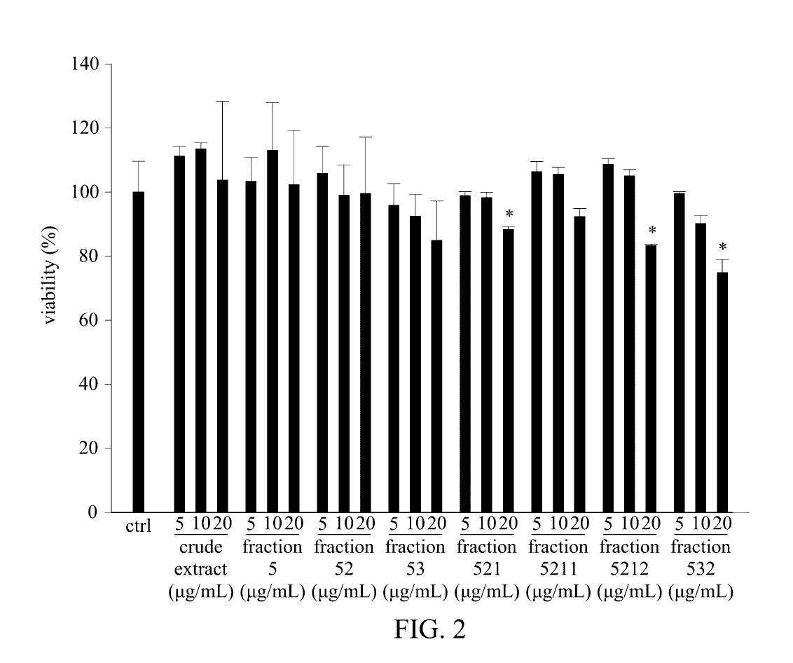

FIG. 2 shows the cytotoxic effects of the crude extract, fractions 5, 52, 53, 521, 5211, 5212 and 532 of M. charantia extracts, on THP-1 cell. The result shows that while the concentrations of the crude extract, fraction 5, fraction 52 and fraction 53, were at 5 μg/mL, 10 μg/mL and 20 μg/mL, no obvious cytotoxic effect was observed. When the concentrations of fraction 5211, fraction 5212 and fraction 532 were at 20 μg/mL, they have effects on the viability of cells, but the viabilities of cells were greater than 75%.

### Example 4

### The Effect of the M. charantia Extract on the Expression of Cytokines Induced by P. gingivalis

### Bacteria Culture

P. gingivalis (ATCC 33277/BCRC 14417) purchased from Bioresource Collection and Research Center of Food Industry Research and Development Institute in Hsinchu. P. gingivalis was incubated with TSB medium in an anaerobic incubator at 37° C. in an atmosphere of 86% of N₂, 10% of CO₂ and 4% of H₂. The TSB medium comprises 15 g of Tryptic Soy broth (Becton, Dicksinson and company, U.S.A.), 2.5 g of yeast extract, 0.5 mL of Hemin and 0.1 mL of Vitamin K dissolved in 500 mL of deionized H₂O.

### Anti-Inflammatory Activity Assay

THP-1 cell line (2×10⁶ cells/mL) cultured by the method described in Example 3 was placed into a 96-well plate, and then 100 μL of RPMI medium without P. gingivalis was added or 0.1% dimethyl sulfoxide (DMSO) containing P. gingivalis with its multiplicity of infection (MOI) of 10 was added as negative controls. Different concentrations of samples from Examples 1 and 2 were added or luteolin (10 μM) was added as positive control. After co-culturing in an incubator at 37° C. in 5% of CO₂ for 24 hours, the supernatants were collected. Then, the levels of IL-6 and IL-8 were analyzed by commercial ELISA kits for IL-6 and IL-8 (Invitrogen).

The term “MOI” is multiplicity of infection which means the number of the bacteria infecting each cell (the number of bacteria/the number of cells). For example, MOI=1 refers to every cell was infected by one bacterial colony.

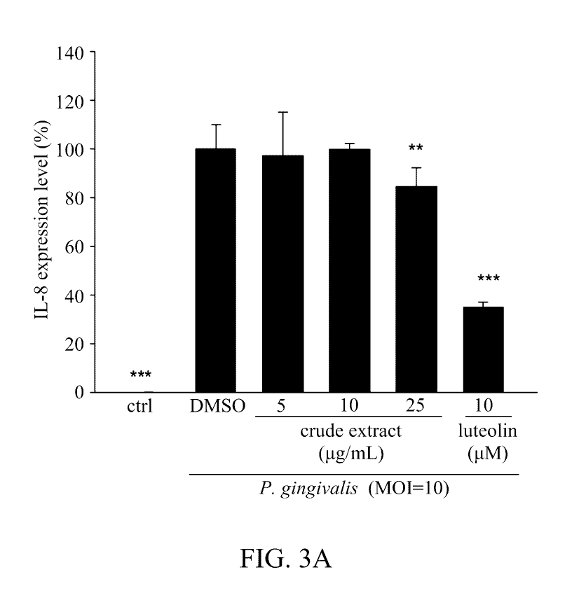

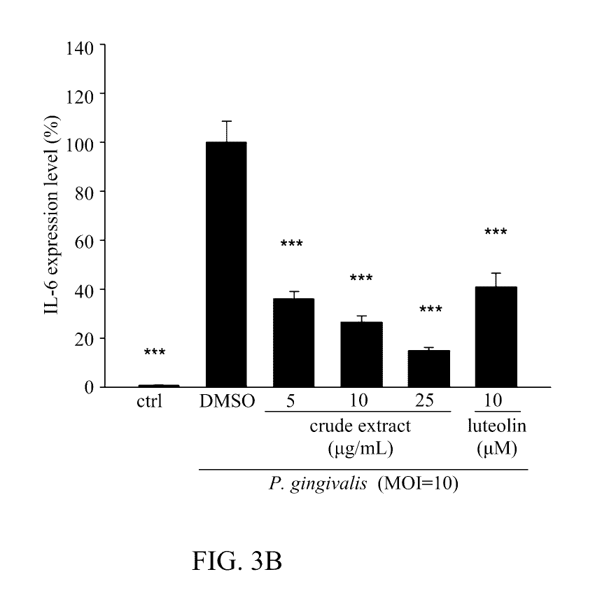

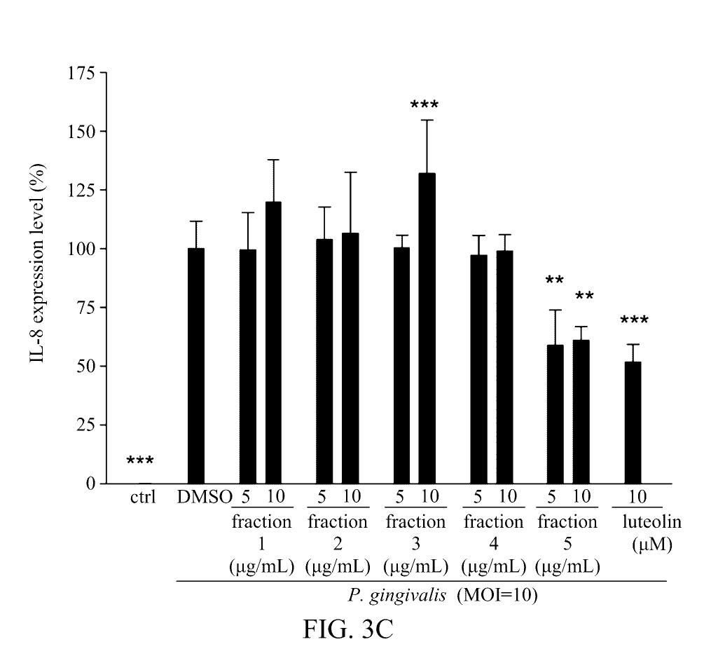

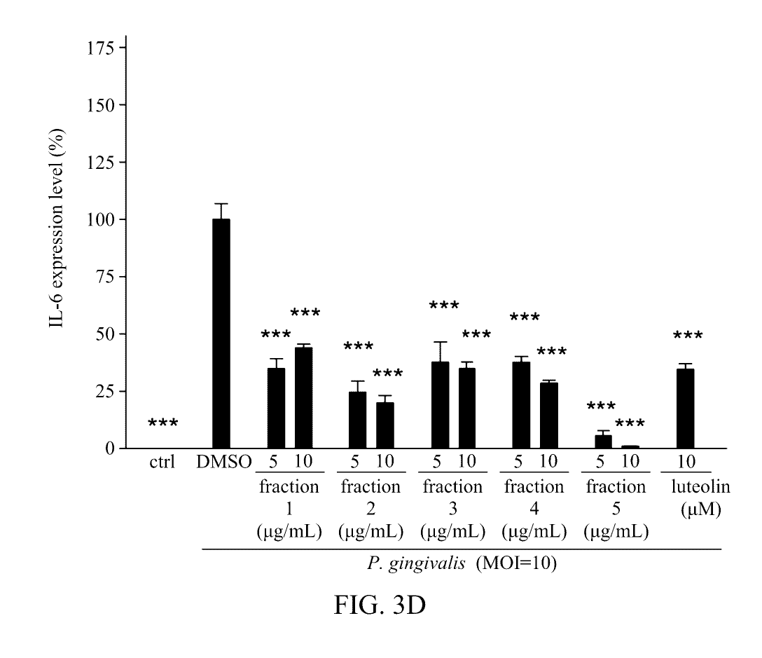

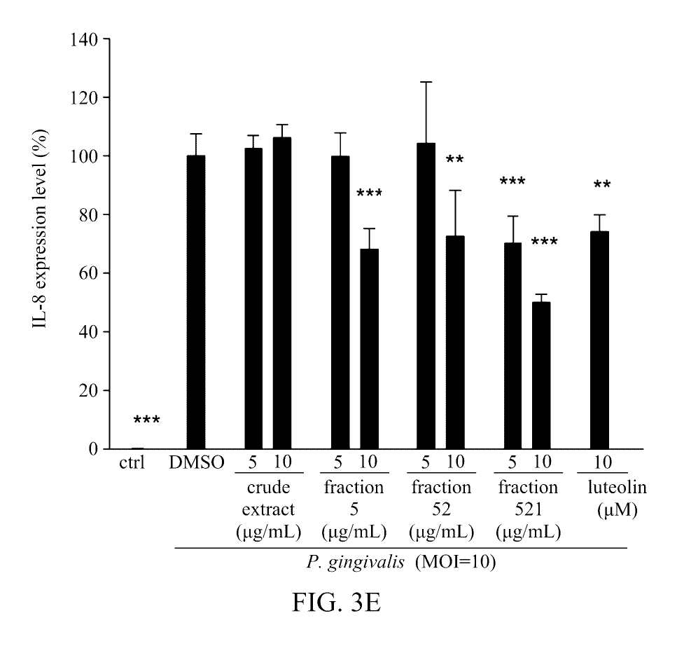

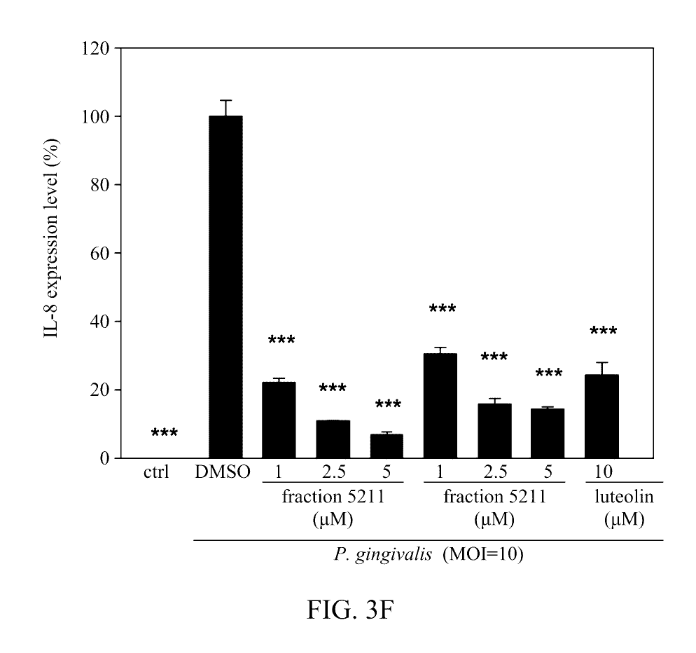

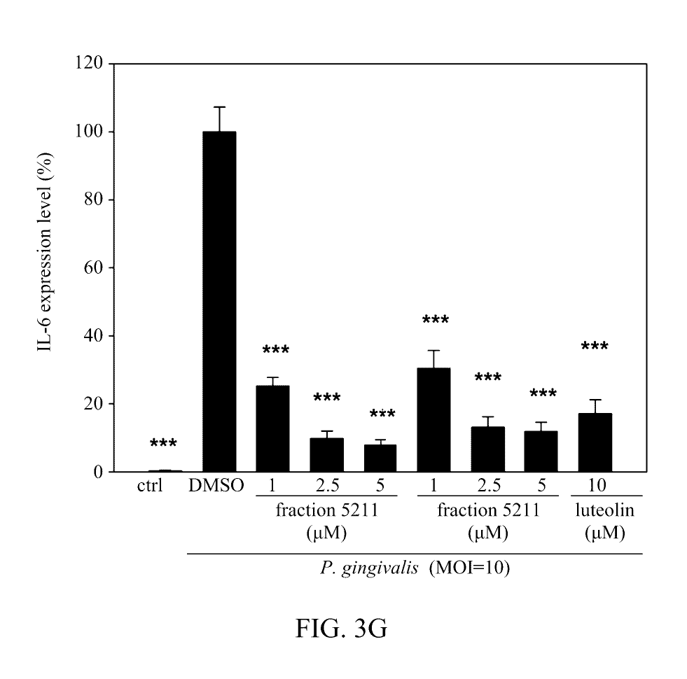

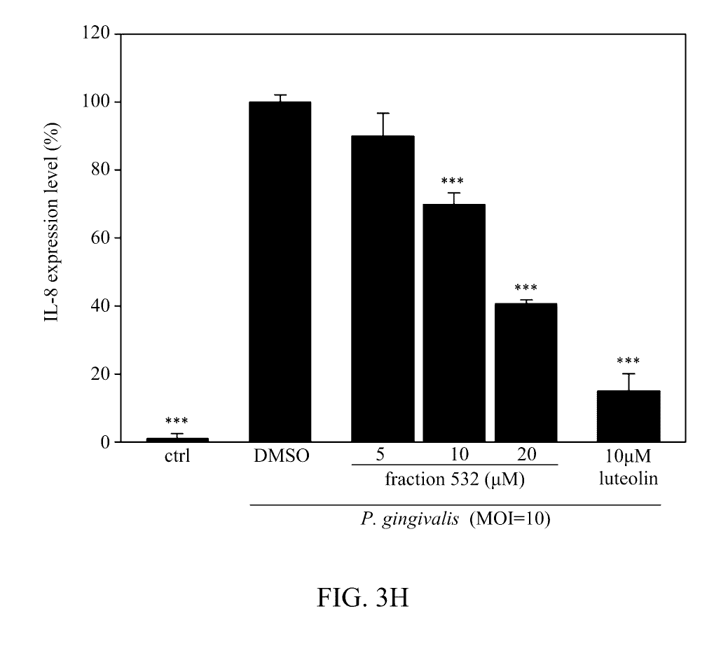

FIGS. 3A to 3I show the effects of different M. charantia extracts on the expressions of cytokines, IL-8 and IL-6, by THP-1 cell lines induced by P. gingivalis, wherein the control group was untreated by P. gingivalis and the extracts.

As shown in FIG. 3A, when the concentration of the crude extract of M. charantia was 25 μg/mL, the expression of IL-8 by THP-1 cell line was obviously inhibited. FIG. 3B shows the effect of the crude extract on the expression of IL-6 by THP-1 cell line. It is found that when the concentration of the crude extract was 5 to 25 μg/mL, the expression of IL-6 induced by P. gingivalis was obviously inhibited.

FIGS. 3C and 3D respectively show the abilities of fraction 1 to fraction 5 in inhibiting the expressions of IL-8 and IL-6 by THP-1 cell lines induced by P. gingivalis. It is discovered that each of the fractions had significant ability to inhibit the expression of IL-6, whereas only fraction 5 shows obvious effect on inhibiting the expression of IL-8.

FIG. 3E shows the abilities of the crude extract, fractions 5, 52 and 521 to inhibit the expression of IL-8 by THP-1 cell lines which were induced by P. gingivalis. As the result of the experiment, the serial fractions further separated from fraction 5 had abilities to inhibit the expression of IL-8 when the concentration of the fraction was 10 μg/mL. Besides, fraction 521 separated from fraction 52 exhibited great inhibition effect at low concentration (5 μg/mL).

FIGS. 3F and 3G respectively show the abilities of fraction 5211 (i.e. 5,19-epoxycucurbita-6,23-diene-3,19,25-triol) and fraction 5212 (i.e. 3,7,25-trihydroxycucurbita-5,23-dien-19-al) in inhibiting the expression of IL-8 and IL-6 by THP-1 cell lines which were induced by P. gingivalis. The results show that 1 μM to 5 μM of fractions 5211 and 5212 are effective on inhibiting the expressions of IL-6 and IL-8 induced by P. gingivalis. The inhibition effects were dose-dependent.

FIGS. 3H and 3I respectively shows the ability of fiction 532 in inhibiting the expressions of IL-8 and IL-6 by THP-1 cell line which was indueced by P. gingivalis. As shown from the result, 5 μM to 20 μM of fraction 532 was effective on inhibiting the expressions of IL-6 and IL-8 by THP-1 cell line.

### Example 5

### The Effects of the M. charantia Extract on Phosphorylation of MAPK Proteins

When TLR2 and TLR4 on the cell membrane are activated by P. gingivalis, MAPK signal transduction proteins, such as ERK, JNK, and p38, would be phosphorylated by a series of signal transduction, thereby stimulating generation of chemokines and proinflammatory cytokines. As a result, chemoraction and inflammation happen.

To analyze the effect of the M. charantia extract on phosphorylation of MAPK proteins, THP-1 cell line cultured by the method described in Example 3 (2×10⁶ cells/mL) was inoculated in 6-cm petridishes. Every 200 μL of cell culture was added 800 μL of fresh media containing DMSO with P. gingivalis (MOI=10), different concentrations of samples from Examples 1 and 2 or luteolin (10 _{N}M) and then co-cultured in a incubator at 37° C. in 5% of CO₂ for 30 mins. Next, the cells were collected and centrifuged to remove the supernatant. After adding phosphate buffer solution (PBS), the collected cells were shook and washed repeatedly for 3 times. Then 100 μL of 1× lysis buffer was added in. The mixture was centrifuged at 12,000 rpm and 4° C. for 15 mins, and the supernatant was collected. The protein was quantified to the identical concentration. After protein electrophoresis was conducted, Western blot was conducted to analyze the level of phosphorylated ERK, JNK and p38 (i.e. p-ERK, p-JNK and p-p38).

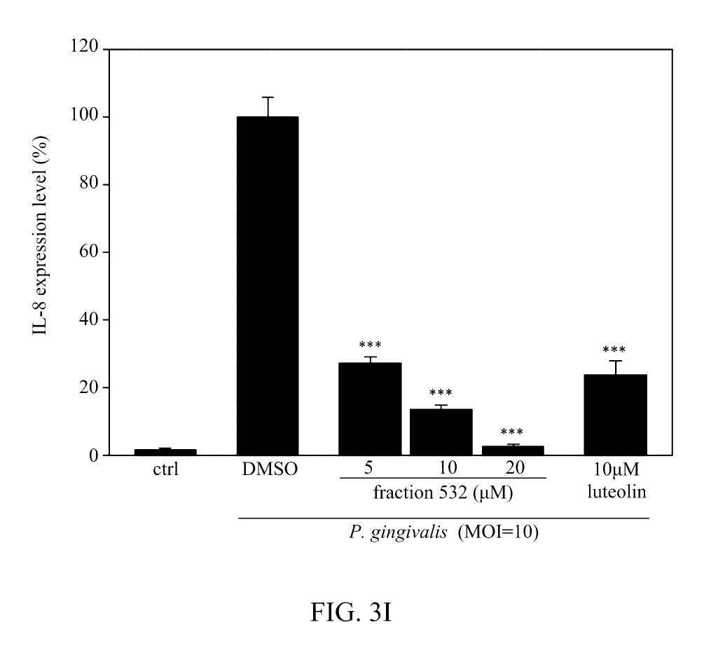

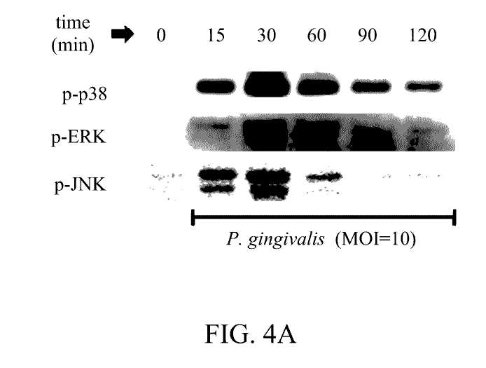

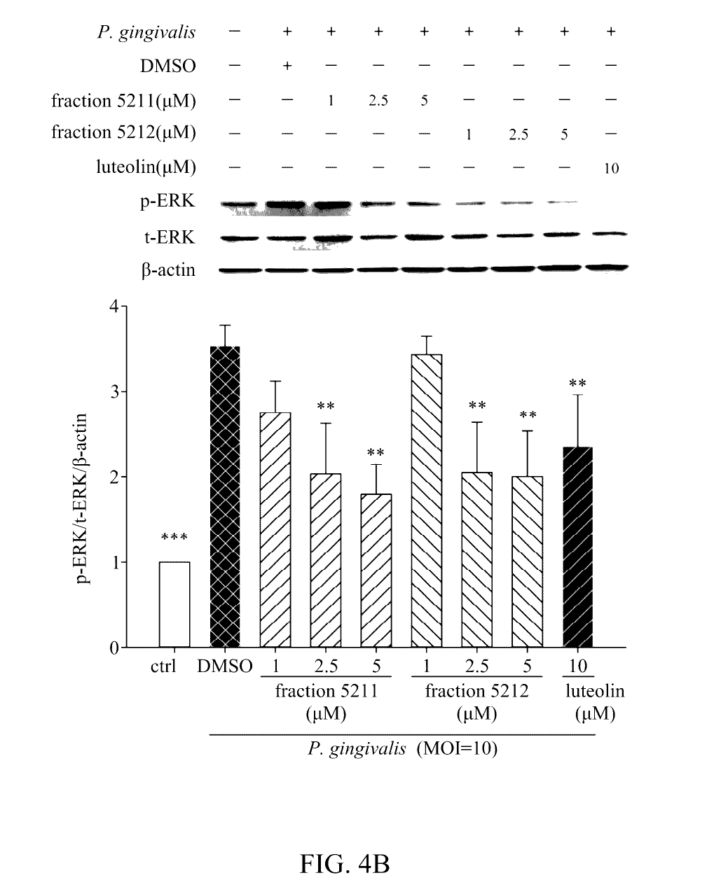

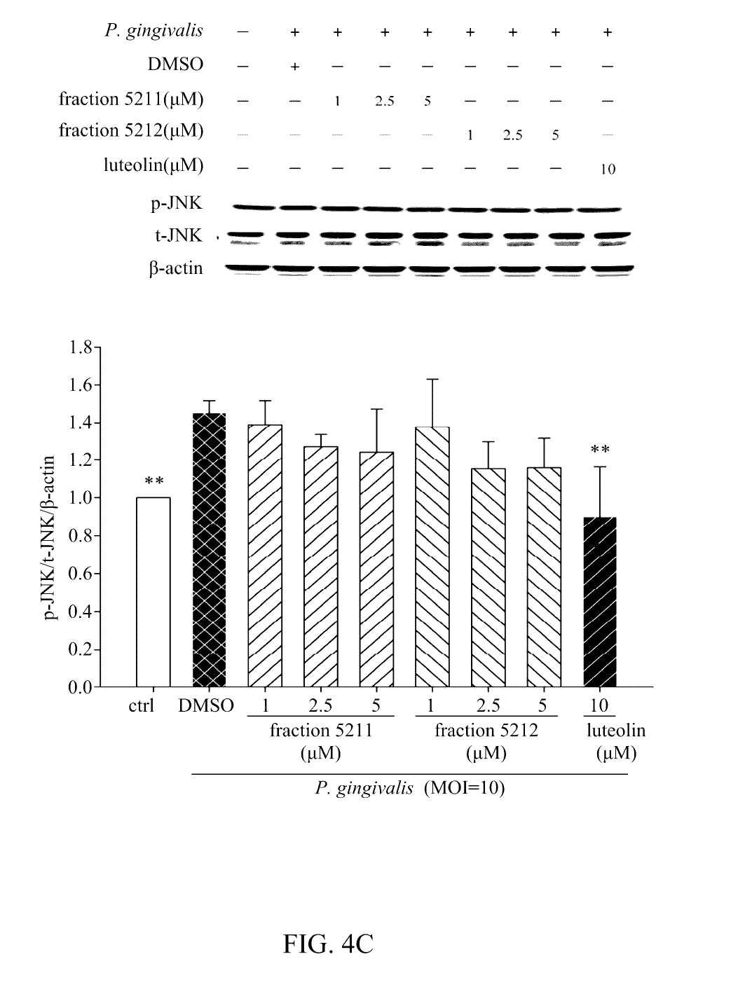

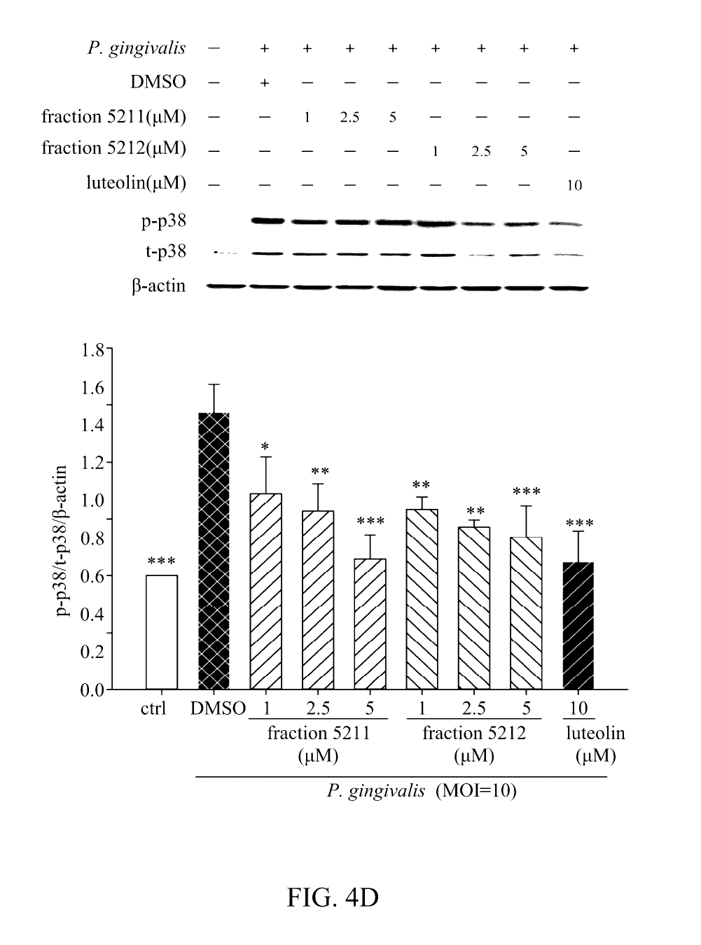

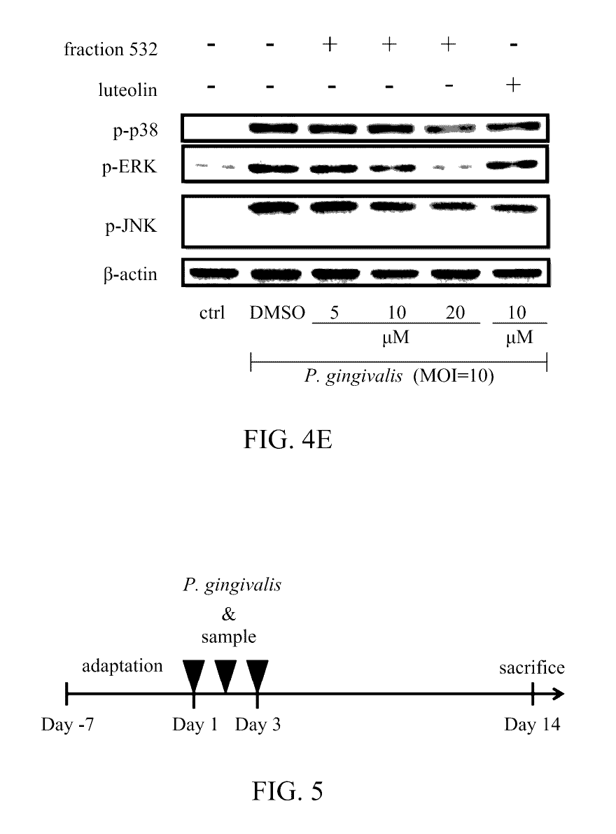

FIG. 4A shows the level of p-ERK, p-JNK and p-p38 in THP-1 cell after co-culturing P. gingivalis and THP-1 cell line for different time periods. It is found that after induced by P. gingivalis for 30 mins, the levels of p-ERK, p-JNK and p-p38 in THP-1 cell reached the peak value and then decreased, wherein compared to the level that did not induced by P. gingivalis (i.e. 0 min), the levels of p-p38 were 5.5-, 12.2-, 6.9-, 3.5-, and 2.4-fold; the levels of p-ERK were 2.0-, 3.4-, 1.5-, 1.1- and 1.3-fold; and the levels of p-JNK were 1.6-, 1.9-, 1.5-, 1.5- and 1.4-fold at 15 min, 30 min, 1 hour and 2 hour.

FIGS. 4B to 4D show that after being induced by P. gingivalis for 30 mins the effects of faction 5211 and fraction 5212 on the levels of p-ERK, p-JNK and p-p38 in THP-1 cell. FIG. 4E shows that after being induced by P. gingivalis for 30 mins the effect of faction 532 on the level of p-ERK. As shown in the FIGs, when fraction 5211 and fraction 5212 were at the concentrations of 1 μM to 5 μM, the phosphorylation of the signal transduction proteins in THP-1 cells was reduced. Moreover, with the increasing of concentration, the effects on inhibiting phosphorylation were greater, which means the inhibition effect was dose-dependent. Likewise, 10 μM and 20 μM of fraction 532 are effective in inhibiting the phosphorylation of ERK.

Previous studies have indicated that the combination of P. gingivalis and complement would activate MAPK signal transduction proteins and thereby initiating immunoreaction [14]. Therefore, the pathogenic bacterium, P. gingivalis, causes the activation of TLR in host, and downstream signal transduction thereof including MAPK pathway. By administration of the M. charantia extract provided by the present invention, the activation of signal transduction proteins therein could be inhibited, so as to block the inflammation of the cell and then to inhibit the expression of the related cytokines effectively.

### Example 6

### Assessment of the Anti-Inflammatory Activity In Vivo

It is known that there is a positive correlation between periodontitis and the expression of COX-2 in gum tissue. When the host was stimulated, which could cause inflammation in promoting the increases of COX-2 and IL-1β.

To assess the effect of kuguacin on anti-inflammation in vivo, 6-week old C57BL/6 male mice were used in the present embodiment. The mice were purchased from Laboratory Animal Center of National Taiwan University, and fed with commercial chow diet and drinking water. After free access of food for 7 days, the mice were grouped for experiments, five animals for each group.

The concentration of P. gingivalis was adjusted to 1×10⁹ CFU/mL by steriled PBS, then centrifuged at 10,000 rpm for 5 min. After removing the supernatant, the pellet was heated in water bath at 80° C. for 30 mins.

According to the method provided in the reference by Molon et al. to establish animal experiment model [15]. Heat-killed P. gingivalis was injected into the gum tissue of C57BL/6 mice to cause inflammation. The mice were anesthetized by ethylether. Samples and bacteria solution were withdrawn using 0.3-mL insulin syringe needle. 10 μL of bacteria and samples were injected into the gum tissue of the mice. If blisters appeared, the injection was successful. As shown in FIG. 5, it shows the flowchart of the injection experiment for three consecutive days, and the first day of the injection was the first day of the experiment.

There were 4 groups in this experiment, steriled PBS injection was the control group; P. gingivalis injection as induced group; P. gingivalis and kuguacin injection as treatment; and luteolin injection as positive control.

After sacrificing the mice at the 14^{th} day of the experiment, the gums of lower jaw of the mice were sampled and the gene expressions of COX-2, IL-6 and TNF-α therein were analyzed. The sequences of the primers used are listed in Table 1.

TABLE 1
The squences of the primers

| SEQ | | | | |
|---|---|---|---|---|
| ID | | | | |
| No. | genes | direction | sequences (5′→3′) | Bp. |
| 1 | TNF-α | F | TTCTGTCCCTTTCACTCACT | 103 |
| 2 | | R | CCTCTTCTGCCAGTTCCA | |
| 3 | COX-2 | F | AAAGAAGGGTTCCCAATT | 184 |
| | | | AAAGAT | |
| 4 | | R | GCATTGAGAGATGGACTGTTAG | |
| 5 | IL-6 | F | GACTGATGCTGGTGACAA | 102 |
| 6 | | R | GTGAAGTGGTATAGACAGGTC | |
| 7 | iNOS | F | AACGCTTCATTCCAATG | 83 |
| 8 | | R | GGCTCTGTTGAGGTCTAA | |
| 9 | GADPH | F | TCCAAGGAGTAAGAAACC | 106 |
| 10 | | R | GAAATTGTGAGGGAGATG | |

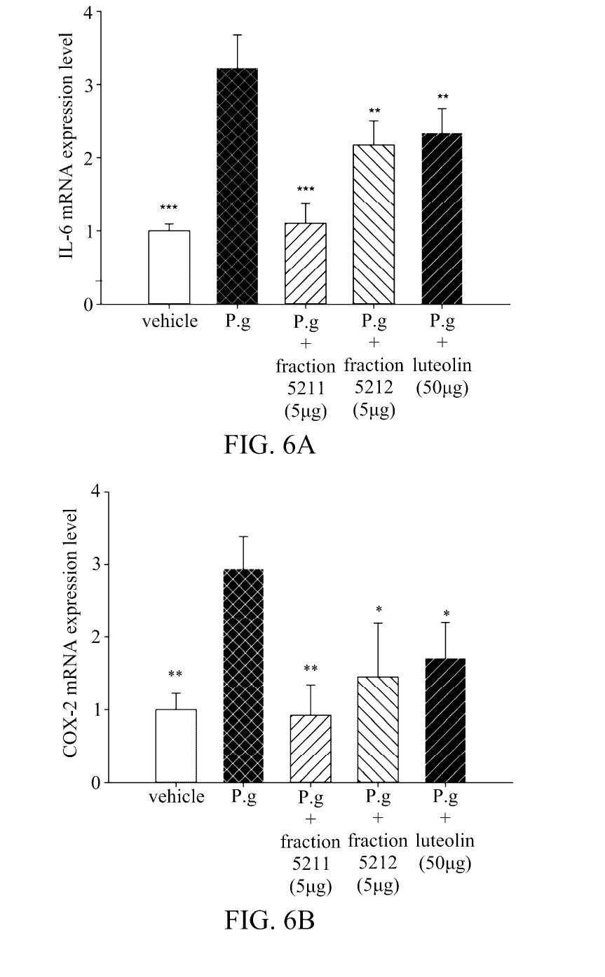

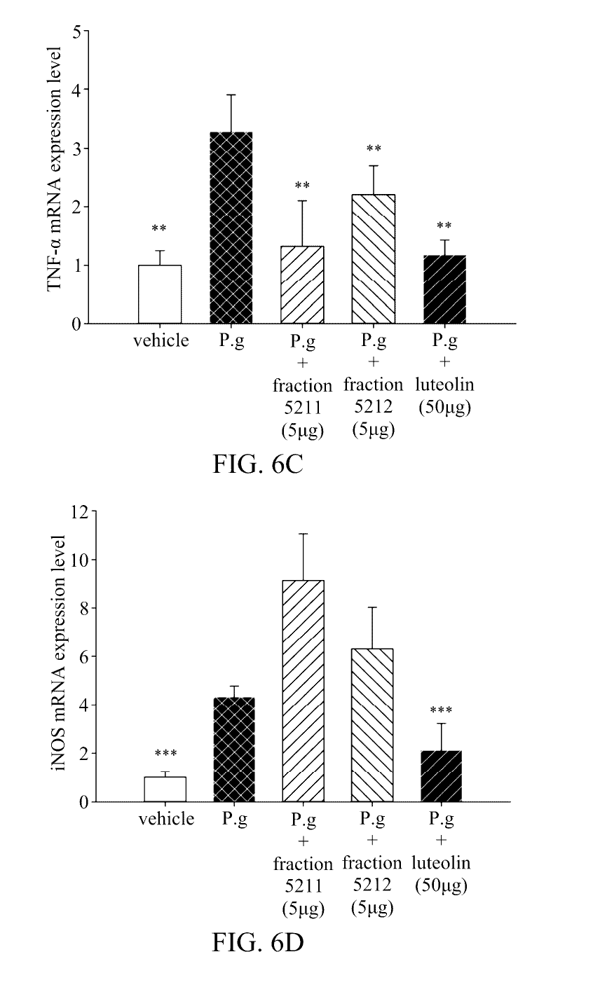

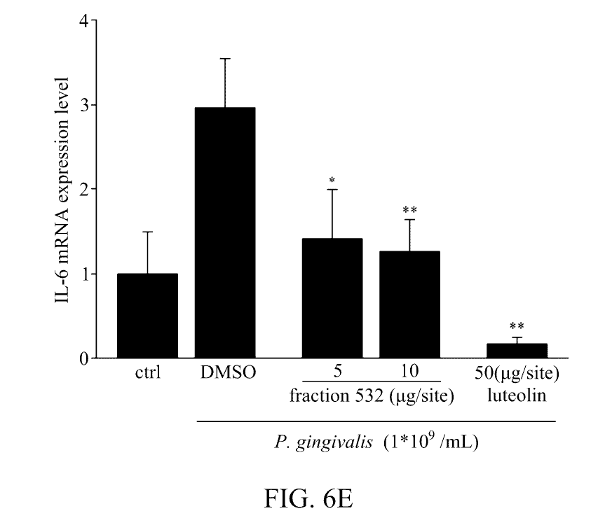

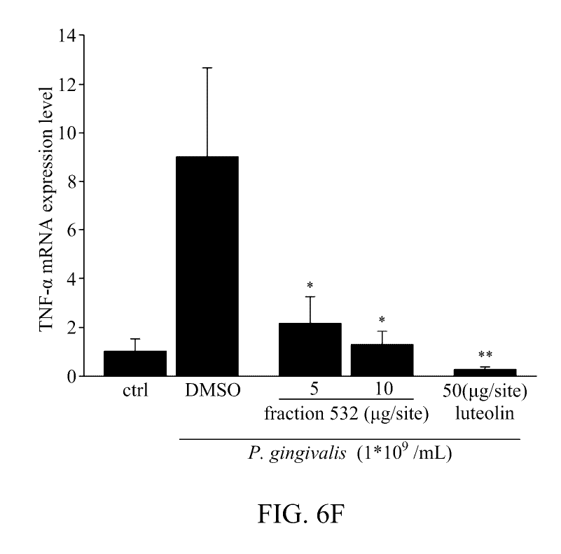

FIGS. 6A to 6D show the effect of kuguacin on the mRNA expression of IL-6, COX-2, TNF-α and iNOS in the gum tissue of the periodontitis C57BL/6 mice model induced by P. gingivalis. The result demonstrates that fraction 5211 and fraction 5212 both are effective in reducing the expression of IL-6, COX-2 and TNF-α. Compared to induced group by P. gingivalis as 100%, the reductions of the expression of each gene by fraction 5211 and fraction 5212 could be calculated as follows: the expressions of IL-6 were reduced to 34% and 67%, respectively; the expression of COX-2 were reduced to 31% and 49%; and the expression of TNF-α were reduced to 40% and 67%.

In the periodontitis animal model, the administration of 5 μg of fraction 5211 and fraction 5212 can reduce the mRNA expressions of IL-6, COX-2 and TNF-α cuased by the P. gingivalis-induced inflammation, and further inhibit the gum tissue breakdown and prevent the subsequent loss of bone substance caused by the activation of osteoclast.

Besides, fraction 5211 and fraction 5212 increase the mRNA expression of iNOS. As suggested in some studies, the increase of iNOS protects the host by guarding against the bacteria in a condition of acute periodontal inflammation. High concentration of NO would reduce the activity of osteoclast and thereby inhibit loss of bone substance [16]. This indicates that fraction 5211 and fraction 5212 can increase the production of NO by promoting the mRNA expression of iNOS and enhance the immunoresponse of gum tissue to against the pathogenic bacteria and to slow down the development of periodontitis to prevent loss of bone sunstance.

FIGS. 6E and 6F shows the effect of fraction 532 on the mRNA expression of IL-6 and TNF-α in the gum tissue of the periodontitis C57BL/6 mice model induced by P. gingivalis.

IL-6 is a cytokine which would greatly express after tissue gets wounded or infected. As shown in the mRNA expression of IL-6, the kuguacin obtained by the method of the present invention has ability to reduce the expression of IL-6 and would be helpful in alleviating inflamed gums caused by the infection of intruded P. gingivalis. Besides, TNF-α and IL-1β are the cytokines expressed significantly when periodontitis occurs, wherein TNF-α is one of the cytokines expressed most significantly after a subject is infected by oral pathogen and related to bone mass biosynthesis [17]. The kuguacin obtained by the method of the present invention has the ability to inhibit the expression of TNF-α, which means the kuguacin contributes to reducing the risk of the loss of bone mass and the loss of tooth.

To sum up, the kuguacin obtained by the method of the present invention can decrease inflammation effectively. The kuguacin can inhibit the phosphorylation of the signal transduction proteins (such as ERK and p38) in the MAPK pathway. Through the animal assay, it can be verified that the kuguacin is able to inhibit the mRNA expressions of COX-2, IL-6, TNF-α, and thereby to inhibit the activation of osteoclast to prevent gum tissue breakdown. Besides, the kuguacin is able to promote the mRNA expression level of iNOS, thereby to increase the production of NO to induce immunoresponse in gum tissue, and can be used to against the pathogen in alleviating the progression of periodontitis thereby to remedy the bone loss of alveolar bone. Since the correlation between peridontitis and many diseases (such as atherosderosis, rheumatoid arthritis, and diabetes) has been demonstrated by several studies, therefore, the extraction of M. charantia obtained by the method of the present invention and the kuguacin comprised therein can not only inhibit peridontitis induced by P. gingivalis, but also prevent or alliviate the related systemic diseases derived from peridontitis.

The principles and effects of the present invention have been described using the above examples, which are not used to limit the present invention. Without departing from the spirit and scope of the present invention, any one skilled in the art can modify the above examples. Therefore, the scope of the present invention should be accorded with the claims appended.

The literatures cited by the present application are listed below, and each of the references is incorporated herein by reference.
1. Pihlstrom, B. L., Michalowicz, B. S., & Johnson, N. W. (2005). Periodontal diseases. The Lancet, 366(9499), 1809-1820.
2. Heitz-Mayfield, L. J., Schätzle, M., Loe, H., Burgin, W., Ånerud, Å., Boysen, H., & Lang, N. P. (2003). Clinical course of chronic periodontitis. Journal of clinical periodontology, 30(10), 902-908.
3. Kozarov, E., & Grbic, J. (2012). Systemic Effects of Periodontal Diseases: Focus on Atherosclerosis. INTECH
4. Nylander, M., Lindahl, T. L., Bengtsson, T., & Grenegård, M. (2008). The periodontal pathogen Porphyromonas gingivalis sensitises human blood platelets to epinephrine. Platelets, 19(5), 352-358.
5. Gomes-Filho, I. S., Passos, J. S., & Seixas da Cruz, S. (2009). Respiratory disease and the role of oral bacteria. Journal of oral microbiology, 2, 158-160.
6. Taylor, G. W. (2001). Bidirectional interrelationships between diabetes and periodontal diseases: an epidemiologic perspective. Annals of periodontology, 6(1), 99-112.
7. HAN, Y. W., FARDINI, Y., CHEN, C., IACAMPO, K. G., PERAINO, V. A., SHAMONKI, J. M., & REDLINE, R. W. (2010). Term Stillbirth Caused by Oral: Fusobacterium nucleatum. Obstetrics and gynecology, 115(2), 442-445.
8. Luo, W., Wang, C. Y., & Jin, L. (2012). Baicalin Downregulates Porphyromonas gingivalis Lipopolysaccharide-Upregulated IL-6 and IL-8 Expression in Human Oral Keratinocytes by Negative Regulation of TLR Signaling. PLoS One, 7(12), e51008-e51008.
9. Holzhausen, M., Spolidorio, L. C., Ellen, R. P., Jobin, M. C., Steinhoff, M., Andrade-Gordon, P., & Vergnolle, N. (2006). Protease-activated receptor-2 activation: a major role in the pathogenesis of Porphyromonas gingivalis infection. The American journal of pathology, 168(4), 1189-1199.
10. Gitlin, J. M., & Loftin, C. D. (2009). Cyclooxygenase-2 inhibition increases lipopolysaccharide-induced atherosclerosis in mice. Cardiovascular Research, 81, 400-407.
11. Gingivitis, P. I. (2004). Treatment of Plaque-induced Gingivitis, Chronic Periodontitis, and Other Clinical Conditions. Journal of Periodontology, 72, 1790-1800.
12. Farhad, S. Z., Aminzadeh, A., Mafi, M., Barekatain, M., Naghney, M., & Ghafari, M. R. (2013). The effect of adjunctive low-dose doxycycline and licorice therapy on gingival crevicular fluid matrix metalloproteinase-8 levels in chronic periodontitis. Dental Research Journal, 10(5), 624-629.
13. Popova, C., & Mlachkova, A. (2009). Effectiveness of additional therapy with NSAID (Aulin) on distribution of shallow and deep periodontal pockets in patients with chronic periodontitis (Pilot study). Journal of IMAB—Annual Proceeding (Scientific Papers), 15(2), 55-57.
14. Hajishengallis, G., & Lambris, J. D. (2011). Microbial manipulation of receptor crosstalk in innate immunity. Nature Reviews Immunology, 11(3), 187-200.
15. de Molon, R. S., de Avila, E. D., Boas Nogueira, A. V., Chaves de Souza, J. A., Avila-Campos, M. J., de Andrade, C. R., & Cirelli, J. A. (2014). Evaluation of the host response in various models of induced periodontal disease in mice. Journal of periodontology, 85(3), 465-477.
16. Ralston, S. H., Ho, L. P., Helfrich, M. H., Grabowski, P. S., Johnston, P. W., & Benjamin, N. (1995). Nitric Oxide: a cytokine-induced regulator of bone resorption. Journal of Bone and Mineral Research, 10(7), 1040-1049.
17. Kesavalu, L., Chandrasekar, B., & Ebersole, J. L. (2002). In vivo induction of proinflammatory cytokines in mouse tissue by Porphyromonas gingivalis and Actinobacillus actinomycetemcomitans. Oral microbiology and immunology, 17(3), 177-180.

What is claimed is:

1. An extracting method for obtaining kuguacin from Momordica charantia, comprising:
providing powder of M. charantia leaves;
extracting a pre-determined weight of the powder of M. charantia leaves with an extracting agent to obtain crude extract thereof;
subjecting the crude extract to a partition extraction process with a first elution solution by column chromatography to obtain a first fraction, wherein the first elution solution is n-hexane or a mixture of more than 0% to 30% by volume of ethyl acetate and 70% to less than 100% by volume of n-hexane;
subjecting the first fraction to a partition extraction process with a second elution solution by column chromatography to obtain a second fraction; and
subjecting the second fraction to a partition extraction process with a third elution solution by column chromatography to obtain a third fraction.

2. The method of claim 1, wherein the extracting agent comprises at least one selected from a group consisting of methanol, ethanol and ethyl acetate.

3. The method of claim 1, wherein the second elution solution is n-hexane or a mixture of more than 0% to 50% by volume of acetone and 50% to less than 100% by volume of n-hexane.

4. The method of claim 1, wherein the third elution solution is a mixture of 20% to 80% by volume of n-hexane and 20% to 80% by volume of acetone.

5. The method of claim 1, wherein the third fraction comprises the kuguacin, and the kuguacin is 5β,19-epoxycucurbita-6,23(E),25(26)-triene-3β,19(R)-diol.

6. The method of claim 1, further comprising subjecting the third fraction to a partition extraction process with a fourth elution solution by column chromatography to obtain a fourth fraction.

7. The method of claim 6, wherein the fourth elution solution is n-hexane or a mixture of 70% to less than 100% of n-hexane and more than 0% to 30% of acetone.

8. The method of claim 6, wherein the fourth fraction comprises the kuguacin, and the kuguacin is 5,19-epoxycucurbita-6,23-diene-3,19,25-triol or 3,7,25-trihydroxycucurbita-5,23-dien-19-al.

9. A pharmaceutical composition, comprising:
a therapeutically effective amount of kuguacin obtained from the method of claim 1; and
a pharmaceutical acceptable carrier.

10. The pharmaceutical composition of claim 9, wherein the therapeutically effective amount of kuguacin is 1 μM to 20 μM.

11. A method of preventing or treating periodontal diseases or diseases caused by Porphyromonas gingivalis in a subject in need thereof, the method comprising administering a medicament comprising kuguacin to the subject.

12. The method of claim 11, wherein the kuguacin is 5β,19-epoxycucurbita-6,23(E),25(26)-triene-3β,19(R)-diol, 5,19-epoxycucurbita-6,23-diene-3,19,25-triol or 3,7,25-trihydroxycucurbita-5,23-dien-19-al.

13. The method of claim 11, wherein the diseases caused by Porphyromonas gingivalis are gingivitis, periodontitis and/or cardiovascular diseases.

14. The method of claim 11, wherein the medicament is used for decreasing the level of cytokine expression.

15. The method of claim 12, wherein the medicament is used for decreasing the level of cytokine expression.

16. The method of claim 13, wherein the medicament is used for decreasing the level of cytokine expression.
